(19) United States
(12) Patent Application Publication
(10) Pub. No.: US 2008/0177390 A1
(43) Pub. Date: Jul. 24, 2008

(54) Disk Replacement Endoprosthesis

(75) Inventors: Steven T. Mitchell, Pleasant Hill, CA (US); Scott A. Yerby, Montara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ST. FRANCIS MEDICAL TECHNOLOGIES, INC., Sunnyvale, CA (US)

(21) Appl. No.: 11/874,845

(22) Filed: Oct. 18, 2007

Related U.S. Application Data
(60) Provisional application No. 60/853,955, filed on Oct. 24, 2006.

(51) Int. Cl. A61F 2/44

(52) U.S. Cl. 623/17.16; 623/17.15

(57) ABSTRACT

A disk replacement endoprosthesis and a procedure for implantation of the endoprosthesis to treat pain, nerve root compression, and neural injury caused by degeneration or injury to vertebral disks. The endoprosthesis may include an intervertebral wedge or other body having a lead-in distractor. A fastening plate may be attached to the intervertebral wedge by a joint such that the first fastening plate may move from a first configuration in which the first fastening plate is in-line with the intervertebral wedge to facilitate insertion of the endoprosthesis to a second configuration in which the first fastening plate is in contact with an anterior surface of the inferior vertebral body for attachment thereto.

### CROSS-REFERENCES TO RELATED APPLICATIONS

This U.S. Provisional Patent application claims the benefit of under 35 U.S.C. §109(e) of U.S. Provisional Patent Application No. 60/853,955, as filed on Oct. 24, 2006, the disclosure of which is incorporated by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

Neck pain affects million of people each year. In many cases, the neck pain may be treated with medication and physical therapy. However, in some cases, these non-surgical treatments do not relieve the pain. One cause of pain can be herniation of a cervical disk in which a rupture or bulge in the cervical disk compresses on nerve roots of the spinal cord. In addition to neck pain, the nerve compression can cause arm pain and serious neurological dysfunction.

Another cause of neck pain may be cervical spinal stenosis (including, but not limited to, central canal and lateral stenosis), and facet arthropathy. In cervical vertebrae, stenosis results in a reduction foraminal area (i.e., the available space for the passage of nerves and blood vessels) which compresses the cervical nerve roots and causes radicular pain. Humpreys, S. C. et al., Flexion and traction effect on C5-C6 foraminal space, Arch. Phys. Med. Rehabil., vol. 79 at 1105 (September 1998). Another symptom of cervical spinal stenosis is myelopathy, which results in neck pain and muscle weakness. Id. Extension and ipsilateral rotation of the neck further reduces the foraminal area and contributes to pain, nerve root compression, and neural injury. Id.; Yoo, J. U. et al., Effect of cervical spine motion on the neuroforaminal dimensions of human cervical spine, Spine, vol. 17 at 1131 (Nov. 10, 1992).

Cervical radiculopathy secondary to disc herniation and cervical spondylotic foraminal stenosis typically affects patients in their fourth and fifth decade and has an annual incidence rate of 83.2 per 100,000 people (based on 1994 information). Cervical radiculopathy is typically treated surgically with either an anterior cervical discectomy and fusion or posterior laminoforaminotomy, with or without facetectomy. Discectomy and fusion is the most commonly performed surgical procedure for cervical radiculopathy, as it has been shown to increase significantly the foraminal dimensions when compared to posterior laminoforaminotomy.

Cervical discectomy and fusion suffers from the disadvantage that fusion of the two adjacent cervical vertebrae leads to a reduction in normal range of motion and increases the amount of stress or wear on the vertebrae adjacent the fusion. As a consequence, there may be accelerated degeneration of the adjacent motion segments. The risk of the need for surgery at adjacent motion segments has been reported to be as high as 30% over ten years. Also, fusion typically requires a bone graft to be taken from the patient's hip. The donor site for the bone graft is a significant source of postoperative pain and other complications. The risk of complications at the donor site has been reported to be as high as 20%.

Disk replacements have been developed as an alternative to fusion. An artificial cervical disk is inserted between adjacent vertebral bodies after discectomy instead of fusing the vertebral bodies together with a bone graft. Disk replacement or cervical disk arthroplasty potentially has the advantage of allowing greater mobility at the treated segment and thus avoiding accelerated degeneration of the adjacent motion segments. Total disk arthroplasty also has the advantage that no bone graft is required. Cervical disk arthroplasty is a difficult and technically challenging invasive surgical procedure. The replacement must be conducted through an anterior approach and current implants require very precise placement and milling of the vertebral bodies to position and secure the implant. Where a precise fit is not achieved, the surgeon converts the procedure to a fusion instead. Because of the complexity of the procedure and the stringent fitment requirements some procedures will be converted to fusion procedures with their attendant disadvantages.

It is desirable to eliminate the need for major surgery for all individuals, and in particular, for the elderly. Accordingly, it would be desirable to have a minimally-invasive cervical disk replacement endoprosthesis that maintains motion and balance while alleviating pain caused by disc herniation, stenosis, and other such conditions caused by damage to, or degeneration of, the cervical spine.

It is also desirable to have an endoprosthesis that is simpler to implant and thus less likely to necessitate reversion to a fusion procedure.

It is further desirable to have minimally-invasive procedures for disk arthroplasty that are less invasive and cause less tissue damage than current technologies and have improved results.

### BRIEF DESCRIPTION OF THE DRAWINGS

Features of the invention, its nature and various advantages will be more apparent from the accompanying drawings and the following detailed description of the preferred embodiments, in which:

FIG. 1A shows a perspective view of a disk replacement endoprosthesis in accordance with one embodiment of the invention;

FIG. 1B shows an anterior view of the disk replacement endoprosthesis of FIG. 1A;

FIG. 1C shows a lateral view of the disk replacement endoprosthesis of FIG. 1A;

FIG. 1D shows an exploded view of the disk replacement endoprosthesis of FIG. 1A illustrating the components;

FIG. 2A shows an anterior view of a disk replacement endoprosthesis in accordance with an alternative embodiment of the invention;

FIG. 2B shows a lateral view of the disk replacement endoprosthesis of FIG. 2A;

FIG. 2C shows an anterior view of a disk replacement endoprosthesis in accordance with an alternative embodiment of the invention;

FIG. 2D shows a lateral view of the disk replacement endoprosthesis of FIG. 2C;

FIG. 3A shows a transverse view of the neck during a procedure to implant the disk replacement endoprosthesis of FIGS. 1A-D illustrating the endoprosthesis in its in-line configuration;

FIG. 3B shows a transverse view of the neck during a procedure to implant the disk replacement endoprosthesis of FIGS. 1A-D illustrating the endoprosthesis in its deployed configuration;

FIG. 3C shows a lateral view of the disk replacement endoprosthesis of FIGS. 1A-D located between adjacent cervical vertebrae in accordance with an embodiment of the invention;

FIG. 3D shows an anterior view of the disk replacement endoprosthesis of FIGS. 1A-D located between adjacent cervical vertebrae after fixation with bone screws in accordance with an embodiment of the invention;

FIG. 4A shows a lateral view of the disk replacement endoprosthesis of FIGS. 2A-B located between adjacent cervical vertebrae in accordance with an embodiment of the invention; and

FIG. 4B shows an anterior view of the disk replacement endoprosthesis of FIGS. 2A-B located between adjacent cervical vertebrae after fixation with bone screws in accordance with an embodiment of the invention;

FIG. 4C shows a lateral view of the disk replacement endoprosthesis of FIGS. 2C-D located between adjacent cervical vertebrae in accordance with an embodiment of the invention; and

FIG. 4D shows a lateral view of the disk replacement endoprosthesis of FIGS. 2C-D illustrating relative movement of the adjacent spinous processes.

### DETAILED DESCRIPTION OF THE INVENTION

In view of the foregoing background of the invention, it is an object of the present invention to provide a minimally-invasive disk replacement endoprosthesis that maintains motion and balance while providing decompression and alleviating pain caused by disc herniation, stenosis, and other such conditions caused by damage to, or degeneration of, the cervical spine.

It is also an object of this invention to provide a disk replacement endoprosthesis that is simpler to implant and thus less likely to necessitate reversion to a fusion procedure.

It is a further object of the present invention to provide minimally-invasive procedures for disk replacement arthroplasty that are less invasive than current technologies and have improved results.

In accordance with the objects and background of the invention, embodiments of the present invention provide for a minimally-invasive surgical implantation method and endoprosthesis for total disk arthroplasty that alleviates pain while preserving motion between adjacent vertebrae. In one embodiment of the present invention an intervertebral endoprosthesis is provided for replacing the disc between a superior vertebral body and an adjacent inferior vertebral body. The endoprosthesis of this embodiment comprises an intervertebral wedge (or other endoprosthesis body) and a fastening plate. The intervertebral wedge and fastening plate each have associated major surfaces that are substantially opposed, so that each can have a generally flattened shape. The intervertebral wedge also has a lead-in distractor to distract the superior vertebral body away from the inferior vertebral body during insertion of the intervertebral wedge. The intervertebral wedge has a lower surface to engage a cephalad surface of the inferior vertebral body in a static position. The intervertebral wedge has a curved upper surface for engaging a caudad surface of the superior vertebral body. The fastening plate is attached to the intervertebral wedge by a joint such that the fastening plate may move from a first configuration in which the fastening plate is in-line with the intervertebral wedge to facilitate insertion of the endoprosthesis to a second configuration in which the fastening plate is in contact with an anterior surface of the inferior vertebral body. The fastening plate is provided with beveled screw holes to allow the fastening plate to be attached to the anterior surface of the inferior vertebral body. In a particular embodiment, the adjacent vertebral bodies are in the cervical region of the spine in other embodiments, the adjacent vertebral bodies are in the thoracic or lumbar regions of the spine.

In one embodiment of the present invention, a procedure is provided for implanting a disc replacement endoprosthesis in a patient. The procedure comprises inserting the endoprosthesis into the patient with the fastening plate in a first configuration in which the fastening plate is in-line with the intervertebral wedge. The surgeon then urges the lead-in distractor on the leading edge of the intervertebral wedge between the adjacent vertebral bodies thereby distracting the superior vertebral body away from the inferior vertebral body and positioning the intervertebral wedge. The surgeon then causes the fastening plate to move from the first configuration into the second configuration adjacent the anterior surface of the inferior vertebral body. The surgeon then fastens the fastening plate to the anterior surface of the inferior vertebral body. In a particular embodiment, the adjacent vertebral bodies are in the cervical spine.

Other implants and methods within the spirit and scope of the invention can be used to relieve pain associated with the neck spine and/or achieve decompression. Additional objects, advantages, and embodiments of the invention are set forth in part in the description which follows, and in part, will be obvious from this description, or may be learned from the practice of the invention. The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the left-most digit of a reference number identifies the drawing in which the reference number first appears.

### Disk Replacement Endoprosthesis

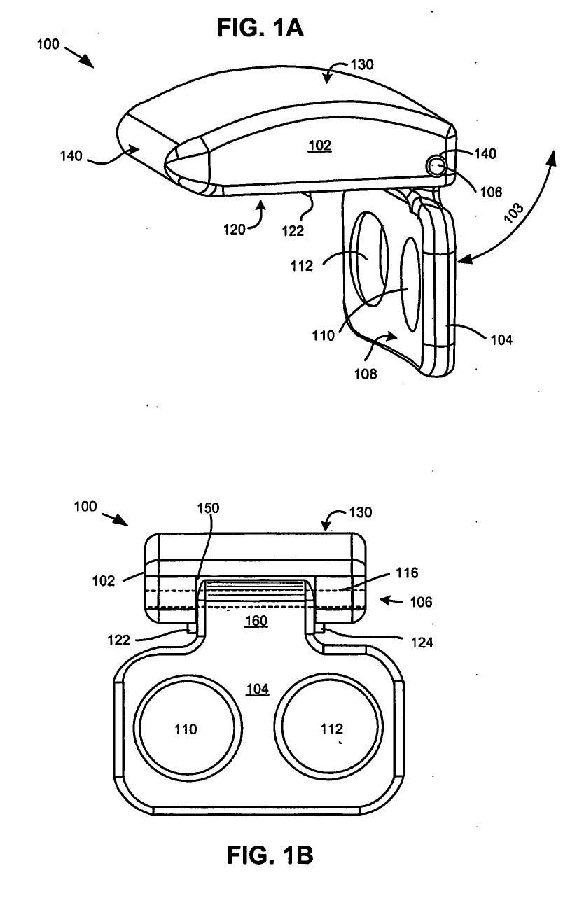

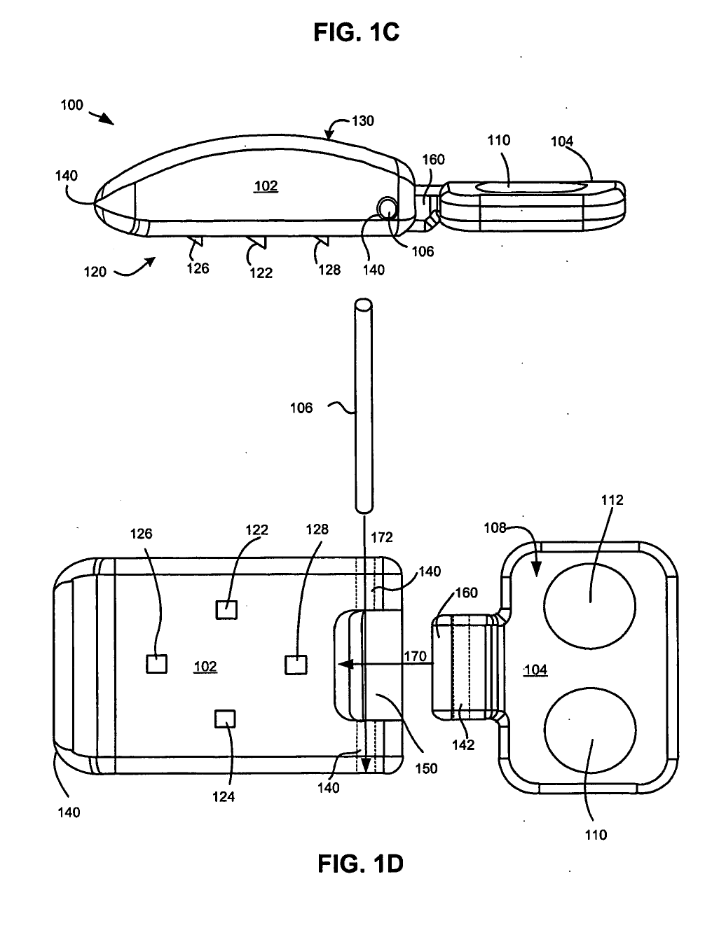

FIG. 1A shows a perspective view of a disk replacement endoprosthesis in accordance with one embodiment of the invention. In this embodiment, the endoprosthesis 100 comprises three parts: intervertebral wedge 102, plate 104, and pin 106. Pin 106 passes through bore 140 and connects intervertebral wedge 102 to plate 104 such that intervertebral wedge 102 may rotate relative to plate 104 around the longitudinal axis of pin 106 as shown by arrow 103.

Plate 104 comprises anterior engagement surface 108 which is generally rectangular in shape and concave to fit against the curved anterior surface of a vertebral body of a cervical vertebrae. The shape and curvature of vertebral body engagement surface 108 should be optimized to fit against the anterior surface of the vertebral body and sized based on the anatomy of the patient. The size of the anterior surface and consequently the size of the plate will depend upon the motion segment to be treated and the size of the patient.

Plate 104 further comprises two bone fastener holes 110, 112. These holes are positioned in plate 104 so as to permit bone fasteners to be inserted through plate 104 into the bone of a cervical vertebral body. In a preferred embodiment, the bone fastener is a bone screw and bone fastener holes 110, 112 are round. However, other fasteners known in the art may be used and plate 104 adapted to be compatible with such fasteners.

Intervertebral wedge 102 comprises lower engagement surface 120 for engaging the surface of the vertebral body inferior to intervertebral wedge 102. Ventral surface 120 is generally rectangular and planar. Lower engagement surface 120 is provided with surface features which promote fixation to a vertebral body inferior to intervertebral wedge 102. In the embodiment shown in FIGS. 1A-D, lower engagement surface 120 comprises four spikes, 122, 124, 126, 128 which aid fixation to a vertebral body (See FIGS. 1A-D). In this embodiment, the spikes are sloped on the face which is closest to lead-in tissue distractor 140 thereby facilitating insertion of endoprosthesis 100. However, the side of spikes 122, 124, 126, 128 closest to plate 104 are vertical this inhibiting intervertebral wedge 102 from sliding out from between the vertebral bodies after implantation. Additional surface treatment such a plasma-spayed titanium may be applied to lower engagement surface 122 to aid fixation to a vertebral body and/or promote bone growth using standard arthroplasty methods and materials. For example, the surfaces of the lower engagement surface 122 can be roughened in order to promote bone ingrowth to stabilize and fix the implant intervertebral wedge 102. In other embodiments, the lower engagement surface 122 can be coated with materials that promote bone growth such as, for example, bone morphogenic protein (“BMP”), or structural materials such as hyaluronic acid “HA,” or other substances which promote growth of bone relative to and into the lower engagement surface 122. The size of the vertebral bodies and consequently the size of the intervertebral wedge will depend upon the motion segment to be treated and the size of the patient.

Intervertebral wedge 102 also comprises upper engagement surface 130 for engaging the caudad surface of the vertebral body superior to intervertebral wedge 102. Upper engagement surface 130 is generally rectangular and convex. Upper engagement surface 130 is a smooth curved surface and is designed to permit some limited motion of the surface of the vertebral body relative to the cephalad surface. In general morphology, in this embodiment, intervertebral wedge 102 forms a segment of a cylinder. The upper engagement surface 130 therefore comprises the cylindrical surface of a segment of a cylinder. The length of intervertebral wedge 102 can be viewed as a chord of the cylinder. The chord subtends an angle in this embodiment of 45 degrees. In other embodiments, the angle may vary between about 25 degree and 60 degrees depending upon the application. Different curvatures may be used based on the particular anatomy to be treated or the range of motion desired or both. In this embodiment the upper engagement surface is smooth to allow relative motion of the superior vertebral body relative to the intervertebral wedge 102.

Intervertebral wedge 102 further comprises lead-in distractor 140. Lead-in distractor 140 is located at distal end 144 of intervertebral wedge 102. Lead-in distractor 140 has a radiused wedge shape which distracts the faces of the adjacent vertebral bodies during introduction of intervertebral wedge 102. Lead-in distractor 140 thereby facilitates introduction of intervertebral wedge 102 between adjacent vertebral bodies during the implantation procedure.

Intervertebral wedge 102 further comprises groove 150. Groove 150 is located at the proximal end 154 of intervertebral wedge 102. Groove 150 is sized so as to receive tongue 160 of plate 104 to allow attachment of plate 104 to intervertebral wedge 102. Groove 150 and tongue 160 are curved so as to allow tongue 160 to rotate within groove 150 over at least 90 degrees of motion as shown by arrow 103 without interference between the plate 104 and intervertebral wedge 102.

FIG. 1B shows an anterior view of endoprosthesis 100 with plate 104 perpendicular to intervertebral wedge 102. Tongue 160 of plate 104 is shown within groove 150 of intervertebral wedge 102. Dotted line 116 shows the position of pin 106 relative to intervertebral wedge 102 and plate 104. Note that bone fastener holes 110, 112 are beveled in this embodiment so that fasteners can be installed flush with the surface of plate 104. Pin 106 sits within a bore that passes through intervertebral wedge 102, groove 150 and tongue 160. Pin 106 may be retained in intervertebral wedge 102 by friction fit or a fastening device. In this embodiment plate 104 rotates freely about pin 106.

FIG. 1C shows a lateral view of endoprosthesis 100. Note that with plate 104 in-line with intervertebral wedge 102, no portion of plate 104 protrudes beyond the lower engagement surface 120 or upper engagement surface 130. This low-profile configuration allows endoprosthesis 100 to be implanted minimally-invasively.

FIG. 1D shows an exploded view of the endoprosthesis 100 illustrating the three major components. Endoprosthesis 100 is assembled by inserting tongue 160 of plate 104 into groove 150 of intervertebral wedge 102 as shown by arrow 170. Pin 106 is then pushed into a tubular bore 140 passing through intervertebral wedge 102 and tubular bore 142 passing through tongue 160 of plate 102. Thus plate 102 is connected to intervertebral wedge 104 in a pivotable manner.

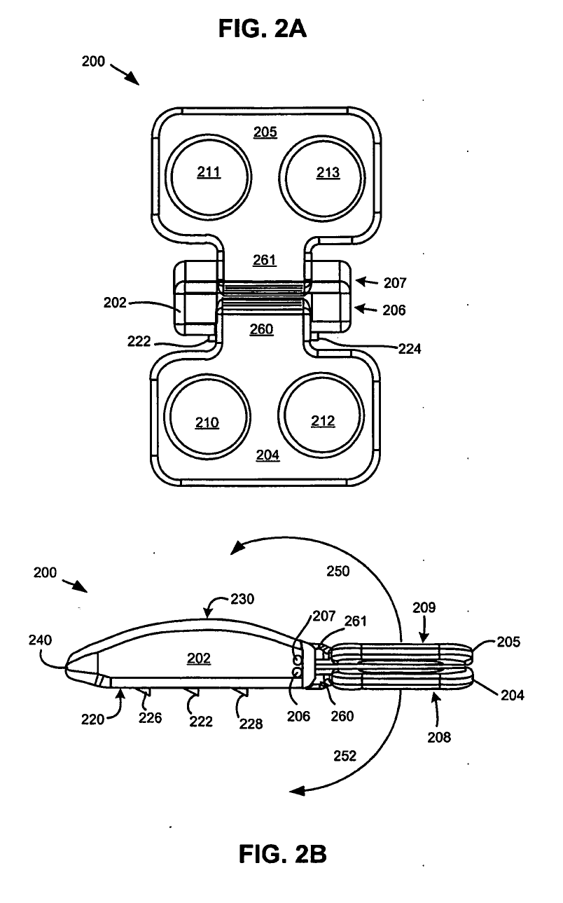

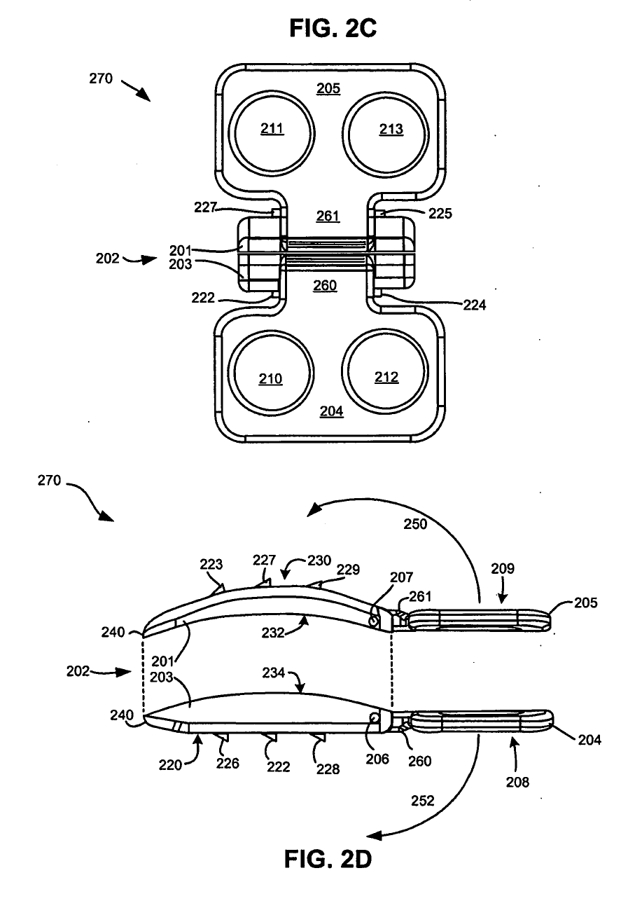

FIG. 2A shows an anterior view of a disk replacement endoprosthesis 200 in accordance with an alternative embodiment of the invention. Endoprosthesis 200 comprises two plates 204, 205 similar in size and shape to plate 104 of endoprosthesis 100. Plate 204 comprises a curved anterior engagement surface 208 for engaging the anterior surface of the vertebral body inferior to intervertebral wedge 202. Plate 205 comprises anterior engagement surface 209 for engaging the anterior surface of the vertebral body superior to intervertebral wedge 202. Intervertebral wedge 202 is of the same general shape as intervertebral wedge 102 of endoprosthesis 100. Intervertebral wedge comprises a lead-in distractor 240, a curved upper engagement surface 230, and a planar lower engagement surface 220 on which are spikes 222, 224, 226, 228. However, groove 252 is sized so as to receive tongue 260 of plate 204 and tongue 261 of plate 205 and allow both to rotate from a low-profile in-line configuration to a position more or less perpendicular to intervertebral wedge 202. The groove is sized so as to allow plate 205 to rotate past perpendicular with intervertebral wedge 202 so as to assume an acute angle with intervertebral wedge 202. Also, two tubular bores pass through the proximal end of intervertebral wedge 202 to allow pins 206, 207 to connect plates 204, 205 respectively.

FIG. 2B shows a lateral view of endoprosthesis 200. Note that with plates 204, 205 in-line with intervertebral wedge 202 as shown, no portion of plates 204, 205 protrudes beyond the lower engagement surface 220 or upper engagement surface 230 of intervertebral wedge 202. This low-profile configuration allows endoprosthesis 200 to be implanted minimally-invasively. After intervertebral wedge 202 has been positioned between adjacent vertebral bodies, plates 204, 205 may be rotated, as shown by arrows 250, 252, to positions approximately perpendicular to intervertebral wedge 202 and in contact with the anterior surfaces of the vertebral bodies above and below intervertebral wedge 202.

FIGS. 2C-D show an alternative embodiment 270 of the endoprosthesis 200 of FIGS. 2A-B in which the intervertebral wedge 202 comprises an upper wedge component 201 and a lower wedge component 203. FIG. 2C shows an anterior view of disk replacement endoprosthesis 270. Endoprosthesis 270 comprises two plates 204, 205 similar in size and shape to plate 104 of endoprosthesis 100. Plate 204 comprises a curved anterior engagement surface 208 for engaging the anterior surface of the vertebral body inferior to intervertebral wedge 202. Plate 205 comprises anterior engagement surface 209 for engaging the anterior surface of the vertebral body superior to intervertebral wedge 202. One tubular bore passes through the proximal end of upper wedge component 201 to allow pin 207 to connect plate 205 to upper wedge component 201. One tubular bore passes through the proximal end of lower wedge component 201 to allow pin 206 to connect plate 204 to lower wedge component 203. Groove 252 passes through upper wedge component 201 and a lower wedge component 203 to receive tongue 260 of plate 204 and tongue 261 of plate 205 and allow both to rotate from a low-profile in-line configuration to a position more or less perpendicular to intervertebral wedge 202. The groove is sized so as to allow plate 205 to rotate past perpendicular with intervertebral wedge 202 so as to assume an acute angle with intervertebral wedge 202.

Intervertebral wedge 202 of endoprosthesis 270 is of the same general shape as intervertebral wedge 202 of endoprosthesis 200. Intervertebral wedge comprises a lead-in distractor 240, a curved upper engagement surface 230, and a planar lower engagement surface 220 on which are spikes 222, 224, 226, 228. In this embodiment, intervertebral wedge 202 comprises an upper wedge component 201 and a lower wedge component 203. In this embodiment, lead-in distractor 240 is comprised in part by the leading edge of upper wedge component 201 and in part by the leading edge of lower wedge component 203. The upper wedge component comprises a plurality of spikes 223, 225, 227, 229 for gripping the vertebral body superior to the intervertebral wedge. In this embodiment, the upper wedge component is designed to be fixed relative to the superior vertebral body. Thus, additional surface treatment such a plasma-spayed titanium may be applied to upper engagement surface 230 to aid fixation to the vertebral body and/or promote bone growth using standard arthroplasty methods and materials. For example, the material of the upper engagement surface 230 can, in this embodiment, be roughened in order to promote bone ingrowth to stabilize and fix the implant intervertebral wedge 202. In other embodiments, the upper engagement surface 230 can be coated with materials that promote bone growth such as, for example, bone morphogenic protein (“BMP”), or structural materials such as hyaluronic acid “HA,” or other substances which promote growth of bone relative to and into the upper engagement surface 230.

FIG. 2D shows a lateral view of endoprosthesis 270 with the upper wedge component 201 separated from the lower wedge component 203. As shown in FIG. 2D, the lower motion surface 232 of upper wedge component 201 is concave, curved and complimentary to the convex upper motion surface 234 of lower wedge component 203. In operation, motion surface 232 is designed to slide over motion surface 234 thereby allowing some freedom of movement at the motion segment. Motion surfaces 232 and 234 are preferably smooth polished surfaces to facilitate relative motion of the surfaces. Motion surfaces 232, 234 may additionally be provided with surface treatments or materials to control or reduce the friction between the surfaces.

Motion surfaces 232, 234, in this embodiment, form a segment of a cylinder. Motion surfaces 232, 234 therefore comprise the cylindrical surface of a segment of a cylinder. The length of intervertebral wedge 102 can be viewed as a chord of the cylinder. The chord subtends an angle in this embodiment of 45 degrees. In other embodiments, the angle may vary between about 25 degree and 60 degrees depending upon the application. Different curvatures may be used based on the particular anatomy to be treated or the range of motion desired or both. In an alternative embodiment, curvature can also be provided from side to side across intervertebral wedge 202. In this alternative embodiment, motion surfaces 232, 234 may comprise portions of a spherical surface. In addition, surfaces 232, 234 may be provided with limit or stop features to control the range of motion at the motion segment.

### Endoprosthesis Implantation Procedure

The intervertebral endoprosthesis of the present invention may be used to replace an intervertebral disk in any region of the spine including the cervical, thoracic and lumbar regions of the spine. In the embodiment disclosed below, a procedure for implantation of the intervertebral endoprosthesis in the cervical region of the spine is disclosed. However, one of skill in the art may readily adapt the disclosed procedure for application in the other regions of the spine without departing from the scope of the present invention.

In one embodiment of the current invention, the standard anterior approach for a cervical discectomy and fusion may be used for implantation of the disk replacement endoprosthesis between adjacent cervical vertebrae. The patient is place in a supine position with the head and neck of the patient supported in a neutral position. Fluoroscopy is used to visualize the spine and standard methods are used to locate the correct disc level. In the anterior approach, a transverse incision is made and a dissection plane is created medially to one side of the esophagus and trachea. Retractors are used to expose the anterior surface of the spinal column. The anterior vertebral muscles are then elevated and retracted to allow access to the intervertebral disc at the correct level. A standard discectomy may then be performed using normal instrumentation and methods to remove the disc material. The end-plate cartilage may be left in place on the end plates however, any bony or soft tissue protrusions that would impede implantation may be removed at this stage. In one embodiment of the present invention, the end-plate cartilage is removed solely from the inferior vertebral body in order to enhance fixation of the lower engagement surface of the intervertebral wedge to the inferior vertebral body.

At this point, if necessary or desired, a standard intradiscal distraction instrument may be used to distract the vertebrae to facilitate introduction of the endoprosthesis. However, the endoprosthesis of the present invention has a lead-in distractor which eliminates or reduces the need for distraction prior to implantation. A properly sized endoprosthesis then may be introduced through the surgical incision. The endoprosthesis is introduced with the fastening plate in-line with the intervertebral wedge. This allows the endoprosthesis to be introduced through a smaller incision resulting in less trauma to the patient, a lower risk of complications and a shorter recovery time. The correct sizing of the endoprosthesis may be achieved either by using a gauge to measure the intervertebral space during the procedure or by using imaging techniques such as a computed tomography scan or magnetic resonance imaging scan to measure the space prior to the procedure. Intervertebral wedges and plates may be provided in a range of sizes to be suitable for a range of different patients. The tongues and grooves of the different intervertebral wedges and plates are preferably all the same size such that any size intervertebral wedge may be used with any one of the plates so as to pick a combination which is best suited to the particular patient's anatomy. Milling of the vertebral bodies may be performed to improve fitment of the endoprosthesis however, it is desirable to avoid any such milling of the bones if possible so as to preserve their physiological integrity.

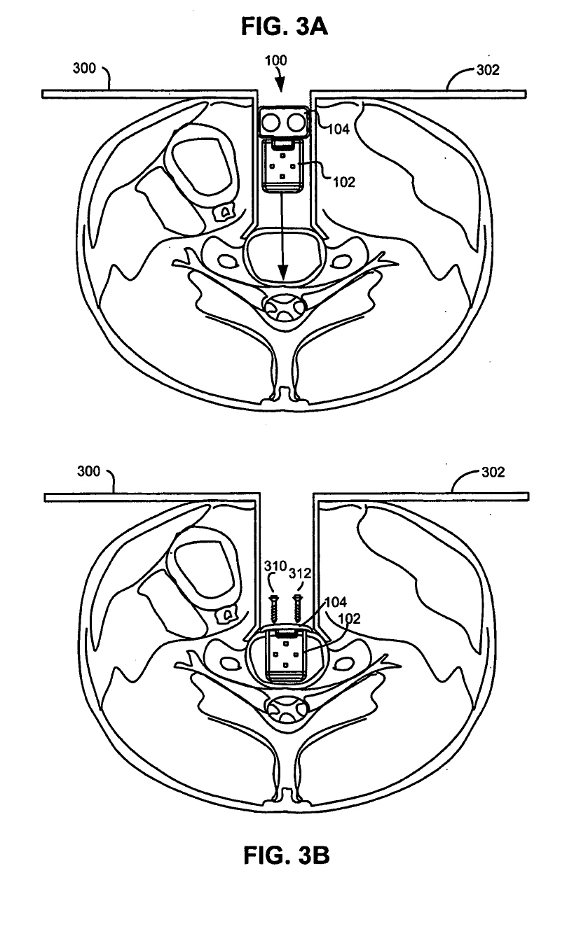

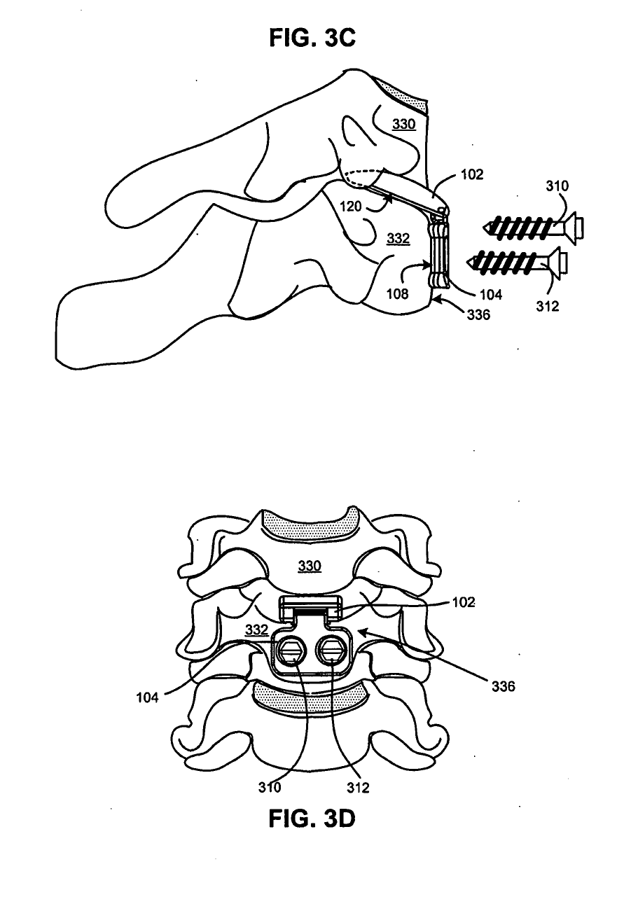

Referring now to FIG. 3A which shows a transverse view of the neck during one embodiment of a procedure to implant the disk replacement endoprosthesis of FIG. 1A. FIG. 3A shows the endoprosthesis in its in-line configuration. As shown in FIG. 3A, in the anterior approach, retractors 300, 302 are used to hold open a port from the front of the neck to the anterior surface of the spinal column. This is a standard method of the anterior approach as used in discectomy and fusion. Note that, as endoprosthesis 100 is introduced into the port, plate 104 is in line with intervertebral wedge 102 and thus presents a lower profile than in its deployed configuration.

The distal end of the endoprosthesis comprises lead-in distractor 140. As the endoprosthesis is introduced between the adjacent vertebral bodies, the lead-in distractor 140 distracts apart the vertebral bodies thereby facilitating introduction of the intervertebral wedge 102. The spikes 122, 124, 126, 128 on the lower engagement surface 220 of the intervertebral wedge 102 are shaped to allow insertion of the endoprosthesis but inhibit removal (See, FIG. 2B). After the intervertebral wedge 102 is properly located between the vertebral bodies as indicated by fluoroscopy of another visualization technique, plate 104 is pushed into contact with the anterior surface of the inferior vertebral body in preparation for fixation.

FIG. 3B shows a transverse view of the neck during one embodiment of a procedure to implant the disk replacement endoprosthesis of FIG. 1A. FIG. 3B illustrates the endoprosthesis in its deployed position. As shown in FIG. 3B, wing 104 is pushed into a position that is almost perpendicular to the intervertebral wedge 102 after intervertebral wedge 102 has been placed in position between adjacent vertebral bodies. This allows anterior engagement surface 108 or plate 104 to come into contact with the anterior surface of the vertebral body inferior to intervertebral wedge 102. The angle between the interdiscal space and the anterior surface of the vertebral body varies from patient to patient. Plate 104 can be positioned at a range of deployment angles relative to intervertebral wedge 102 to compensate for these variations in anatomy and still make good contact with the anterior surface of the inferior vertebral body. The angle between plate 104 and intervertebral wedge 104 will thus vary somewhat from perpendicular. When anterior engagement surface 108 comes into contact with the anterior surface of the vertebral body inferior to intervertebral wedge 102, it may be secured into position with fasteners. In this embodiment, bone screws 310, 312 are screwed into the vertebral body through bone fastener holes 110, 112.

FIG. 3C shows a lateral view of the disk replacement endoprosthesis of FIG. 1A located between adjacent cervical vertebrae in accordance with an embodiment of the invention. In this view, intervertebral wedge 102 is shown in position between superior vertebral body 330 and inferior vertebral body 332. Plate 104 is shown in its deployed position in contact with the anterior surface 336 of the inferior vertebral body 332. Note that plate 104 is now in position for insertion of bone screws 310, 312 through bone fastener holes 110, 112 into inferior vertebral body 332. In this view, it can be seen that the angle between the lower engagement surface 120 of intervertebral wedge 102 and the anterior engagement surface 108 of plate 104 is somewhat larger than 90 degrees because of the anatomy of the patient.

FIG. 3D shows an anterior view of the disk replacement endoprosthesis of FIG. 1A located between adjacent cervical vertebrae after fixation with bone screws in accordance with an embodiment of the invention. In this view, bone screws 310, 312 are shown after insertion through bone fastener holes 110, 112 into inferior vertebral body 332. Depending on the type of fastener used, hole may be drilled into inferior vertebral body 332 prior to insertion of the fasteners in order to facilitate insertion of the fasteners. Bone screws 310, 312 are preferably sized such that after insertion, the heads of bone screws 310, 312 are flush with the anterior surface of plate 104.

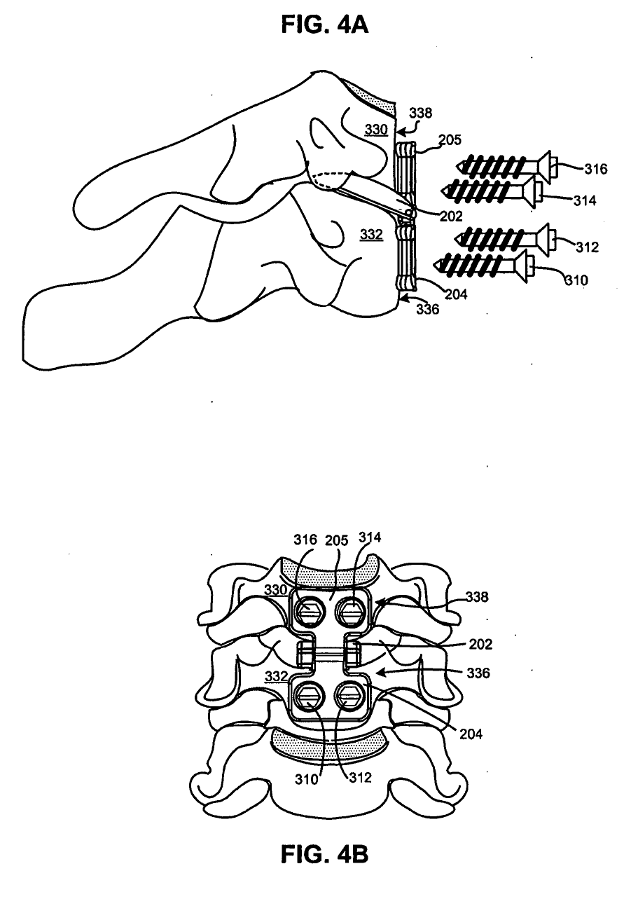

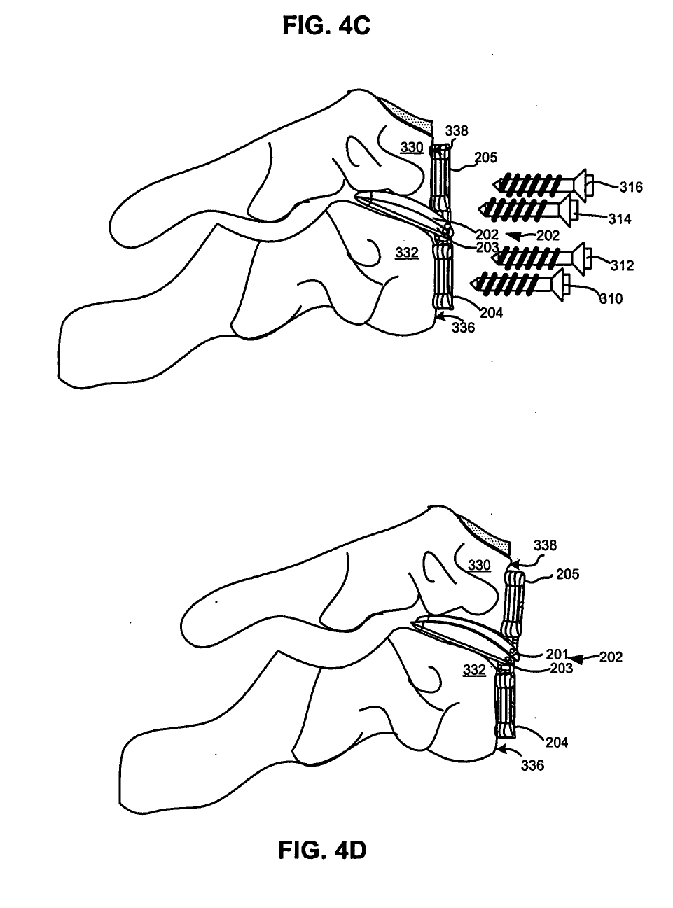

Referring now to FIG. 4A, endoprosthesis 200 is implanted in the same manner as endoprosthesis 100. The endoprosthesis 200 is inserted through the anterior approach port in its low-profile configuration with plates 204, 205 in-line with intervertebral wedge 202 as shown in FIG. 2B. After intervertebral wedge 202 has been positioned between the adjacent vertebral bodies, plates 204, 205 are pivoted into contact with the anterior surfaces of the inferior and superior vertebral bodies 332 and 330 respectively. FIG. 4A shows a lateral view of the disk replacement endoprosthesis of FIG. 2A located between adjacent cervical vertebrae in accordance with an embodiment of the invention. In this view, intervertebral wedge 202 is shown in position between superior vertebral body 330 and inferior vertebral body 332. Plate 204 is shown in its deployed position in contact with the anterior surface 336 of the inferior vertebral body 332. Note that plate 204 is now in position for insertion of bone screws 310, 312 through bone fastener holes 110, 112 into inferior vertebral body 332. Plate 205 is shown in its deployed position in contact with the anterior surface 338 of the superior vertebral body 330. Note that plate 205 is now in position for insertion of bone screws 314, 316 through bone fastener holes 211, 213 into superior vertebral body 330. As shown in FIG. 4A, plate 205 is deployed past perpendicular with intervertebral wedge 202.

FIG. 4B shows an anterior view of the disk replacement endoprosthesis of FIG. 2A located between adjacent cervical vertebrae after fixation with bone screws in accordance with an embodiment of the invention. In this view, bone screws 310, 312, 314, 316 are shown after insertion through bone fastener holes 210, 212, 211, 213 into inferior vertebral body 332 and superior vertebral body 330. Depending on the type of fastener used, holes may be drilled into inferior vertebral body 332 and superior vertebral body 330 prior to insertion of the fasteners in order to facilitate insertion of the fasteners. Bone screws 310, 312, 314, 316 are preferably sized such that after insertion, the heads of bone screws 310, 312, 314, 316 are flush with the anterior surfaces of plates 204 and 205.

Referring now to FIG. 4C, endoprosthesis 270 is implanted in the same manner as endoprosthesis 200. The endoprosthesis 270 is inserted through the anterior approach port in its low-profile configuration with plates 204, 205 in-line with intervertebral wedge 202 as shown in FIG. 2D. After intervertebral wedge 202 has been positioned between the adjacent vertebral bodies, plates 204, 205 are pivoted into contact with the anterior surfaces of the inferior and superior vertebral bodies 332 and 330 respectively. FIG. 4C shows a lateral view of the disk replacement endoprosthesis of FIGS. 2C-D located between adjacent cervical vertebrae in accordance with an embodiment of the invention. In this view, intervertebral wedge 202 is shown in position between superior vertebral body 330 and inferior vertebral body 332. Plate 204 is shown in its deployed position in contact with the anterior surface 336 of the inferior vertebral body 332. Note that plate 204 is now in position for insertion of bone screws 310, 312 through bone fastener holes 110, 112 into inferior vertebral body 332. Plate 205 is shown in its deployed position in contact with the anterior surface 338 of the superior vertebral body 330. Note that plate 205 is now in position for insertion of bone screws 314, 316 through bone fastener holes 211, 213 into superior vertebral body 330. As shown in FIG. 4C, plate 205 is deployed past perpendicular with intervertebral wedge 202.

FIG. 4D shows another lateral view of the endoprosthesis of FIG. 4C. As shown in FIG. 4C, upper wedge component 201 may slide relative to lower wedge component 203 to provide some range of movement at the motion segment. This design has the advantage that the topology of the surface dividing upper wedge component 201 from lower wedge component 203 defines the range of motion which may be selected so as to control the relative motion of the upper and lower vertebra without independent of the geometry or condition of the surfaces of the vertebral body. Also, in this view, bone screws 310, 312, 314, 316 are shown after insertion through bone fastener holes 210, 212, 211, 213 into inferior vertebral body 332 and superior vertebral body 330. Depending on the type of fastener used, holes may be drilled into inferior vertebral body 332 and superior vertebral body 330 prior to insertion of the fasteners in order to facilitate insertion of the fasteners. Bone screws 310, 312, 314, 316 are sized such that, after insertion, the heads of bone screws 310, 312, 314, 316 are flush with the anterior surfaces of plates 204 and 205.

After implantation of the endoprosthesis of the present invention, proper positioning of the disk replacement endoprosthesis is verified with fluoroscopic or similar visualization. When proper placement has been verified, then the anterior approach port is closed using the standard surgical technique.

### Endoprosthesis Materials

In some embodiments, the disk replacement endoprosthesis can be fabricated from medical grade metals such as titanium, stainless steel, cobalt chrome, and alloys thereof, or other suitable implantable materials having similar high strength and biocompatible properties. Additionally, the endoprosthesis can be at least partially fabricated from a shape memory metal, for example Nitinol, which is a combination of titanium and nickel. Such materials are typically radiopaque, and appear during x-ray imaging, and other types of imaging.

Plates 104, 204, 205 and pins 106, 206, 207 are preferably made of a durable implantable metal material such as titanium, titanium alloy, cobalt-chromium-molybdenum, cobalt-chromium or stainless steel. The anterior engagement surfaces 108, 208, 209 of plates 104, 204, 205 may be treated to facilitate fixation to the anterior surfaces of the cervical vertebral bodies. The surfaces may, for example, be provided with a porous titanium surface, plasma-spayed titanium or similar surface that promotes bone growth and enhances fixation of the plate wedge to the vertebral body. The anterior engagement surfaces 108, 208, 209 of plates 104, 204, 205 may also, be provided with surface features, such as roughening or spikes to enhance fixation. The other surfaces of plates 108, 208, 209 are preferably smooth and radiussed to reduce trauma to surrounding tissues.

Intervertebral wedges 102, 202 may be made from the same implantable metal materials as plates 104, 204, 205. The lower engagement surface 120 or 122 may be treated as described above to enhance fixation to the inferior vertebral body and/or encourage bone growth to enhance fixation. The upper engagement surfaces 13, 230 of intervertebral wedges 102, 202 should be smooth and radiussed to allow for some motion of the superior vertebral body relative to the intervertebral wedge.

The intervertebral wedge may be composed in whole or in part of natural bone or synthetic bone. In some circumstances the bone material allows for better fixation to adjacent vertebral bodies. Furthermore, each surface of the endoprostheses which is desired to be fixed in position relative to a vertebral body can comprise a porous surface to promote bone ingrowth and fixation. One such treatment can be with plasma spray titanium, and another, with a coating of sintered beads. Alternatively, the surface may be formed with cast porous surfaces, where the porous surface is integral to the endoprosthesis. As a further alternative, the surfaces can be roughened in order to promote bone ingrowth. In other embodiments, the surfaces can be coated with materials that promote bone growth such as for example bone morphogenic protein (“BMP”), or structural materials such as hyaluronic acid (“HA”), or other substances which promote growth of bone on other external surfaces of the endoprosthesis. These measures facilitate fixation of the endoprosthesis surface where desired to the vertebral body but do not result in fusion of the joint, thereby retaining spinal mobility, while also accomplishing replacement of the disc and distraction of the vertebrae.

One of the physiological functions of an intervertebral disk is to act as a shock absorber for the spine. In order to reproduce this physiological function, endoprostheses 100, 200 and/or portions thereof and in particular intervertebral wedges 102, 202 may also be fabricated from somewhat flexible and/or deflectable material. In these embodiments, the endoprosthesis and/or portions thereof can be fabricated in whole or in part from medical grade biocompatible polymers, copolymers, blends, and composites of polymers. A copolymer is a polymer derived from more than one species of monomer. A polymer composite is a heterogeneous combination of two or more materials, wherein the constituents are not miscible, and therefore exhibit an interface between one another. A polymer blend is a macroscopically homogeneous mixture of two or more different species of polymer. Many polymers, copolymers, blends, and composites of polymers are radiolucent and do not appear during x-ray or other types of imaging. However, fluoroscopic visualization may be necessary to enable proper placement of the endoprosthesis. Thus, radio-opaque markers may be applied to the surface of the endoprosthesis or radio-opaque materials may be included in the polymer composite.

One group of biocompatible polymers is the polyaryl ester ketones which has several members including polyetheretherketone (PEEK), and polyetherketoneketone (PEKK). PEEK is proven as a durable material for endoprosthesis, and meets the criterion of biocompatibility. Medical grade PEEK is available from Victrex Corporation of Lancashire, Great Britain under the product name PEEK-OPTIMA. Medical grade PEKK is available from Oxford Performance Materials under the name OXPEKK, and also from CoorsTek under the name BioPEKK. These medical grade materials are also available as reinforced polymer resins, such reinforced resins displaying even greater material strength. In an embodiment, the endoprosthesis can be fabricated from PEEK 450G, which is an unfilled PEEK approved for medical implantation available from Victrex. Other sources of this material include Gharda located in Panoli, India. PEEK 450G has the following approximate properties:

| Property | Value |
|---|---|
| Density | 1.3 g/cc |
| Rockwell M | 99 |
| Rockwell R | 126 |
| Tensile Strength | 97 MPa |
| Modulus of Elasticity | 3.5 GPa |
| Flexural Modulus | 4.1 GPa |

PEEK 450G has appropriate physical and mechanical properties and is suitable for carrying and spreading a physical load between the adjacent spinous processes. The endoprosthesis and/or portions thereof can be formed by extrusion, injection, compression molding and/or machining techniques.

It should be noted that the material selected can also be filled. Fillers can be added to a polymer, copolymer, polymer blend, or polymer composite to reinforce a polymeric material. Fillers are added to modify properties such as mechanical, optical, and thermal properties. For example, carbon fibers can be added to reinforce polymers mechanically to enhance strength for certain uses, such as for load bearing devices. In some embodiments, other grades of PEEK are available and contemplated for use in endoprosthesis in accordance with the present invention, such as 30% glass-filled or 30% carbon-filled grades, provided such materials are cleared for use in implantable devices by the FDA, or other regulatory body. Glass-filled PEEK reduces the expansion rate and increases the flexural modulus of PEEK relative to unfilled PEEK. The resulting product is known to be ideal for improved strength, stiffness, or stability. Carbon-filled PEEK is known to have enhanced compressive strength and stiffness, and a lower expansion rate relative to unfilled PEEK. Carbon-filled PEEK also offers wear resistance and load carrying capability.

As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. As mentioned, the endoprosthesis can be comprised of polyetherketoneketone (PEKK). Other materials that can be used include polyetherketone (PEK), polyetherketoneetherketoneketone (PEKEKK), polyetheretherketoneketone (PEEKK), and generally a polyaryletheretherketone. Further, other polyketones can be used as well as other thermoplastics. Reference can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.” Other materials such as Bionate7, polycarbonate urethane, available from the Polymer Technology Group, Berkeley, Calif., may also be appropriate because of their good oxidative stability, biocompatibility, mechanical strength and abrasion resistance. Other thermoplastic materials and other high molecular weight polymers can also be used.

A disk replacement endoprosthesis and a procedure for implantation of the endoprosthesis to treat pain, nerve root compression, and neural injury caused by degeneration or injury to vertebral disks are described hereinabove. The endoprosthesis may eliminate and/or relieve complications inherent in standard fusion procedures. The procedure may also avoid the need for milling of the vertebral bodies necessitated by replacement disks thereby reducing trauma caused by implantation and simplifying the procedure. The combination of endoprosthesis and procedure may thereby facilitate discectomy without fusion and improve patient outcome. A particular embodiment is suitable for cervical disk replacement.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. In particular, the intervertebral endoprosthesis of the present invention may be used in all regions of the spine including the cervical, thoracic and lumbar regions. It is intended that the scope of the invention be defined by the claims and their equivalents.

What is claimed is:

1. An endoprosthesis for replacing a disc between a superior vertebral body and an adjacent inferior vertebral body, the endoprosthesis comprising an intervertebral wedge and a first fastening plate wherein:
the intervertebral wedge has a lead-in distractor to distract the superior vertebral body away from the inferior vertebral body during insertion of the intervertebral wedge;
the first fastening plate is attached to the intervertebral wedge by a joint such that the first fastening plate may move from a first configuration in which the first fastening plate is in-line with the intervertebral wedge to facilitate insertion of the endoprosthesis to a second configuration in which the first fastening plate is in contact with an anterior surface of the inferior vertebral body; and
the first fastening plate is adapted to be attached to the anterior surface of the inferior vertebral body.

2. The endoprosthesis of claim 1 wherein:
the intervertebral wedge has a lower wedge surface to engage a cephalad surface of the inferior vertebral body in a static position;
the intervertebral wedge has a curved upper wedge surface to engage a caudad surface of the superior vertebral body;

3. The endoprosthesis of claim 2, wherein:
the endoprosthesis comprises a second fastening plate attached to the intervertebral wedge by a joint such that the second fastening plate may move from a first configuration in which the second fastening plate is in-line with the intervertebral wedge to facilitate insertion of the endoprosthesis to a second configuration in which the second fastening plate is in contact with an anterior surface of the superior vertebral body; and
the second fastening plate is adapted to be attached to the anterior surface of the superior vertebral body.

4. The endoprosthesis of claim 1 wherein the intervertebral wedge comprises an upper wedge component which includes the upper wedge surface and a lower wedge component which includes the lower wedge surface and wherein the upper wedge component is designed to slidingly engage the lower wedge component so as to permit motion between the inferior vertebral body and the superior vertebral body.

5. The endoprosthesis of claim 4 wherein the upper wedge component and lower wedge component form a plain bearing.

6. The endoprosthesis of claim 2 wherein the curved upper surface comprises features to engage the caudad surface of the superior vertebral body in a static position.

7. The endoprosthesis of claim 6 wherein the upper wedge surface can move with respect to the lower wedge surface so as to permit motion between the inferior vertebral body and the superior vertebral body.

8. The endoprosthesis of claim 1 wherein the first fastening plate comprises a concave surface adapted to engage the anterior surface of the inferior vertebral body.

9. The endoprosthesis of claim 3 wherein the second fastening plate comprises a concave surface adapted to engage the anterior surface of the superior vertebral body.

10. An endoprosthesis for replacing a disc between a superior vertebral body and an adjacent inferior vertebral body, the endoprosthesis comprising an intervertebral wedge and a first fastening plate wherein:
the intervertebral wedge has a lead-in distractor to distract the superior vertebral body away from the inferior vertebral body during insertion of the intervertebral wedge, a lower wedge surface to engage a cephalad surface of the inferior vertebral body, and an upper wedge surface to engage a caudad surface of the superior vertebral body; and
the first fastening plate is attached to the intervertebral wedge by a joint such that the first fastening plate may move between a first configuration in which the first fastening plate is in-line with the intervertebral wedge and a second configuration in which the first fastening plate is about perpendicular to the intervertebral wedge.

11. The endoprosthesis of claim 10 wherein the lower wedge surface comprises surface features adapted to prevent movement of the lower wedge surface with respect to the inferior vertebral body.

12. The endoprosthesis of claim 11 wherein the upper wedge surface comprises surface features adapted to prevent movement of the upper wedge surface with respect to the superior vertebral body.

13. The endoprosthesis of claim 12 wherein the upper wedge surface can move relative to the lower wedge surface.

14. The endoprosthesis of claim 10 wherein:
the endoprosthesis comprises a second fastening plate attached to the intervertebral wedge by a joint such that the second fastening plate may move from a first configuration in which the first fastening plate is in-line with the intervertebral wedge and a second configuration in which the first fastening plate is about perpendicular to the intervertebral wedge.

15. A procedure for implantation in a patient of an endoprosthesis for replacing a disc between a superior vertebral body and an adjacent inferior vertebral body, wherein the endoprosthesis comprises an intervertebral wedge and a fastening plate wherein the intervertebral wedge has a lead-in distractor at its distal end and wherein the fastening plate is attached to the intervertebral wedge by a joint such that the fastening plate may move from a first configuration in which the fastening plate is in-line with the intervertebral wedge to facilitate insertion of the endoprosthesis to a second configuration in which the fastening plate is about perpendicular to the intervertebral wedge wherein the method comprises:
a) inserting the endoprosthesis into the patient with the fastening plate in the first configuration;
b) urging the lead-in distractor between the adjacent superior vertebral body and inferior vertebral body thereby distracting the superior vertebral body away from the inferior vertebral body;
c) causing the fastening plate to move from the first configuration into the second configuration; and
d) fastening the fastening plate to the anterior surface of the inferior vertebral body.

16. The procedure of claim 15 wherein the endoprosthesis comprises a second fastening plate wherein the second fastening plate is attached to the intervertebral wedge by a joint such that the second fastening plate may move from a first configuration in which the fastening plate is in-line with the intervertebral wedge to facilitate insertion of the endoprosthesis to a second configuration in which the fastening plate is about perpendicular to the intervertebral wedge wherein the method comprises:
c1) causing the second fastening plate to move from the first configuration in to the second configuration; and
d1) fastening the second fastening plate to the anterior surface of the superior vertebral body.

17. The procedure of claim 16 comprising:
d) fastening the fastening plate with a bone screw to the anterior surface of the inferior vertebral body; and
d1) fastening the second fastening plate with a bone screw to the anterior surface of the superior vertebral body.

18. An endoprosthesis for replacing a disc between a first vertebral body and an adjacent second vertebral body, the endoprosthesis comprising:
an intervertebral body having substantially opposed major surfaces and a lead-in distractor extending along a first edge of the major surfaces so as to distract the first vertebral relative to the second vertebral body during insertion of the intervertebral endoprosthesis;
a first fastening plate having opposed major surfaces;
a fastener extendable from the fastening plate so as to attached the fastening plate to an anterior surface of the first vertebral body;
a pivotal joint coupling the fastening plate to the intervertebral body such that the first fastening plate may move from a first configuration to a second configuration, the fastening plate in the first configuration in-line with the intervertebral body to facilitate insertion of the endoprosthesis, the fastening plate in the second configuration angled so as to contact with the anterior surface.

19. A procedure for implantation in a patient of an endoprosthesis for replacing a disc between a superior vertebral body and an adjacent inferior vertebral body, the method comprising:
a) urging a lead-in distractor of the endoprosthesis between the adjacent superior vertebral body and inferior vertebral body so as to distract the superior vertebral body and the inferior vertebral body away from each other;
b) advancing a body of the endoprosthesis behind the lead-in distractor and between the distracted vertebral bodies, while a fastening plate of the endoprosthesis extends from the endoprosthesis body in-line with the endoprosthesis body so as to define a first configuration;
c) moving the fastening plate from the first configuration into a second configuration extending adjacent an anterior surface of one of the vertebral bodies by articulating a pivotal joint between the endoprosthesis body and the fastening plate; and
d) fastening the fastening plate to the anterior surface of the adjacent vertebral body.
